(19) United States
(12) Patent Application Publication
(10) Pub. No.: US 2020/0113483 A1
(43) Pub. Date: Apr. 16, 2020

(54) SYSTEM AND METHOD FOR MONITORING BREATHING AND MOVEMENT

(71) Applicant: IDA Health, Inc., Berg Kampenhout (BE)

(72) Inventors: Philippe LANGE, Jehay (BE); David Lawrence CAMP, JR., Berg Kampenhout (BE); Giovanni AMOROSO, Haarlem (NL)

(21) Appl. No.: 16/158,628

(22) Filed: Oct. 12, 2018

(51) Int. Cl. A61B 5/113; A61B 5/00; A61B 5/08

(52) U.S. Cl. CPC A61B 5/113; A61B 5/4818; A61B 5/0826; A61B 5/0816; A61B 5/02444; A61B 5/6804; C08K 2201/001; A61B 5/6823; A61B 5/4815

(57) ABSTRACT

A device for monitoring breathing or movement of a living being. The device includes a sensor having a conductive elastomer having a variable resistance and a textile engaged to the sensor.

### CROSS-REFERENCE TO RELATED APPLICATION

n/a

### FIELD

The present technology is generally related to methods and systems for measuring breathing and movement of living beings.

### BACKGROUND

Respiratory abnormalities are the symptoms of numerous diseases and maladies. Such maladies include, for example, sleep apnea, Sudden Infant Death Syndrome (SIDS), or accidental suffocation, among many others. More than 18 million American adults have sleep apnea, in which breathing repeatedly stops and starts during sleep. SIDS is commonly known as the unexplained sudden death of an infant under one year of age. In 2016, there were about 1,500 cases of SIDS in the United States and about 900 deaths to accidental suffocation and strangulation in bed. A SIDS death occurs quickly and is often associated with sleep, with no signs of suffering.

During sleep, an adult or infant can experience a lack of oxygen and/or excessive carbon dioxide levels. The body has the ability to compensate for insufficient oxygen and/or excess carbon dioxide by increasing breathing or exhalation accordingly, which in turn can change the body's movement. As such, certain types of irregularities in an infant or an adult's breathing activity can be an indicator of SIDS, the likelihood of SIDS, or the presence of sleep apnea, among other respiratory conditions.

Current methodologies to measure breathing changes in a patient, however, are bulky and unwieldy. For example, whole-body plethysmography is used to measure respiratory parameters in conscious unrestrained patients but requires patients to be constrained in a phone-booth sized enclosure or requires large and constricting equipment.

### SUMMARY

The techniques of this disclosure generally relate to a system, device, and method for measuring and monitoring movement and breathing in a living being.

In one aspect, the present disclosure provides a device for monitoring breathing or movement of a living being. The device includes a sensor having a conductive elastomer having a variable resistance and a textile engaged to the sensor.

In another aspect, the conductive elastomer planar.

In another aspect, the conductive elastomer is embedded within the textile.

In another aspect, the sensor defines a first end and a second end opposite the first end, and wherein the sensor includes an electrical connector, and wherein the first end and the second end are disposed within the electrical connector.

In another aspect, the sensor includes a second conductive elastomer having a variable resistance different than the conductive elastomer.

In another aspect, the textile defines a length, width, and height, and wherein the conductive elastomer and the second conductive elastomer are each at least one from the group consisting of: disposed entirely at the same height within the textile and disposed at entirely at different heights within the textile.

In another aspect, the device further includes a non-conductive material enclosing the conductive elastomer.

In another aspect, the textile is at least one form the group consisting of a garment, a bed sheet, and a patch including an adhesive configure to be removeably adhered to skin of the living being.

In another aspect, the variable resistance of the conductive elastomer is between 1 kohms and 100 kohms.

In another aspect, the conductive elastomer changes resistance when the conductive elastomer is deformed.

In one aspect, a medical system for monitoring breathing or movement of a living being having a body includes a sensor having including a conductive elastomer having a variable resistance. A textile is engaged to the sensor. A controller is in communication with the sensor, and is configured to, in real time, measure changes in a resistance of the conductive elastomer and correlate the measured changes in the resistance of the conductive elastomer to at least one from the group consisting of breathing and movement of the living being when at least a portion of the body of the living being applies a force to the textile without direct contact to the sensor.

In another aspect, the controller is further configured to identify a breathing pattern based on the measured changes in the resistance of the conductive elastomer, compare the identified breathing pattern to a plurality predetermined abnormal breathing patterns, and if the identified breathing pattern corresponds to one of the plurality of predetermined abnormal breathing patterns, generate an alert.

In another aspect, the controller includes a wireless communication transmitter/receiver configured to communicate with a remote controller.

In another aspect, the conductive elastomer is planar.

In another aspect, the variable resistance of the conductive elastomer is between 1 kohms and 100 kohms.

In another aspect, the sensor define a first end and a second end opposite the first end, and wherein the sensor includes an electrical connector, and wherein the first end and the second end are disposed within the electrical connector.

In another aspect, the sensor includes a second conductive elastomer having a variable resistance different than the conductive elastomer.

In another aspect, the controller is integral with the textile.

In another aspect, the textile is at least one form the group consisting of a garment, a bed sheet, and a patch including an adhesive configure to be removeably adhered to skin of the living being.

In one aspect, a medical system for monitoring breathing or movement of a living being having a body includes a sensor including a conductive elastomer having a variable resistance between 1 kohms and 100 kohms. a textile, the sensor being enclosed within the textile. The sensor defines a first end and a second end opposite the first end, and the sensor includes an electrical connector extending away from the textile, and the first end and the second end are disposed within the electrical connector. A controller is in communication with the sensor and configured to receive the electrical connector, the controller being configured to, in real time: measure changes in the resistance of the conductive elastomer, correlate the measured changes in the resistance of the conductive elastomer to at least one from the group consisting of breathing and movement of the living being when the body of the living being applies a force to the textile without direct contact to the sensor, identify a breathing pattern based on the measured changes in the resistance of the conductive elastomer, compare the identified breathing pattern to a plurality predetermined abnormal breathing patterns, and if the identified breathing pattern corresponds to one of the plurality of predetermined abnormal breathing patterns, generate an alert.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a top slice view of a movement and breathing detection system constructed in accordance with the principles of the present application;

FIG. 2 is a side cross-sectional view the sensor and textile of FIG. 1;

FIG. 3 is a top slice view of another movement and breathing detection system including two conductive elastomers constructed in accordance with the principles of the present application;

FIG. 4 is a side cross-sectional view the sensor and textile of FIG. 3;

FIG. 5 is a top slice view of another movement and breathing detection system constructed in accordance with the principles of the present application;

FIG. 6 is a side cross-sectional view the sensor and textile of FIG. 5;

FIG. 7 is a side-cross sectional view of another embodiment of the sensor and textile shown in FIG. 1;

FIG. 8 is a front view of an infant atop an exemplary movement and breathing detection system and a computing device in communication with the system and having a displaying showing measured movements over a 10 second time frame;

FIG. 9 is the view of FIG. 8 at a different 10 second time frame showing different movements of the infant;

FIG. 10 is the view of FIG. 9 at a different 10 second time frame showing different movements of the infant;

FIG. 11 is a side view a pregnant woman having an exemplary movement and breathing detection system disposed within her undergarment; and

FIG. 12 is a side view of a patient in a wheelchair having an exemplary movement and breathing detection system disposed within the seat of the wheelchair.

### DETAILED DESCRIPTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

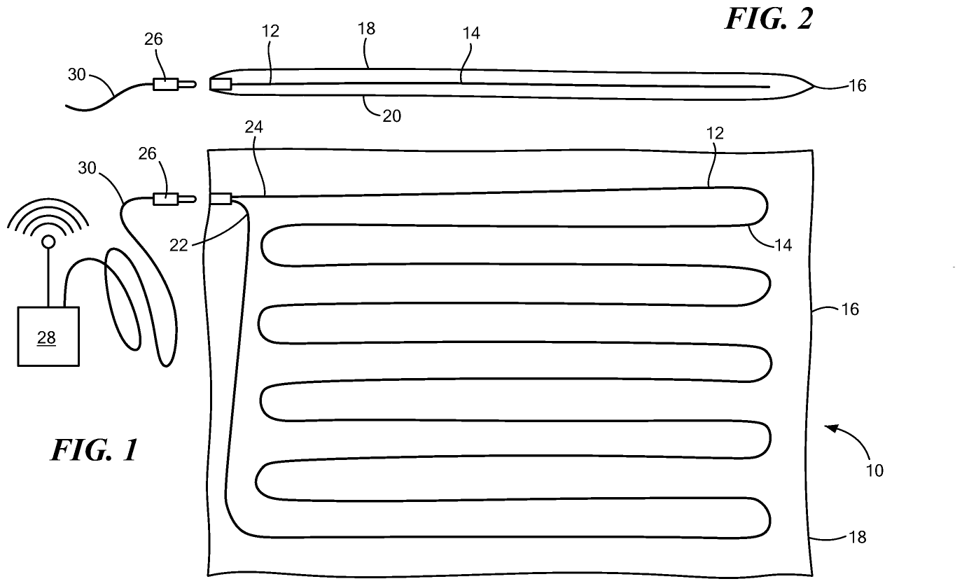

Referring now to the drawings in which like designators refer to like elements, there is shown in FIGS. 1 and 2 an exemplary breathing and movement measurement system constructed in accordance with the principles of the present application and designated generally as “10.” The system 10 includes a sensor 12 configured to measure movement and/or breathing of a living being, for example, a human, animal, or in utero human or animal, when a deformation force is applied to the sensor 12. The sensor 12 includes a conductive elastomer 14 having a variable resistance ranging from 2 kohms and 100 kohms depending on the degree of deformation. In an exemplary configuration, the elastomer 14 includes a thermoplastic material and a semiconductor, such as carbon nanotubes, graphite, or silicon, or a conductor, such as manganese, dispersed within. However, the elastomer 14 having a variable resistance described herein is not limited to any one composition so long as it exhibits suitable piezoresistive and/or conductive properties. Further examples of the elastomer 14, and the process of manufacturing the same, may be found in WIPO Patent Publication No.: WO/2015/049067, the entirety of which is expressly incorporated by reference herein. The elastomer 14 may define any shape or size. For example, the elastomer 14 may define a wire shape having a sold or hollow cross-section, or may alternatively define planar shape, such as a mat, and may be folded or manipulated to define a predetermined shape or a fit within a predetermined volume.

In the configuration shown in FIG. 1, the elastomer 14 defines a serpentine shape and is engaged with a textile 16. In particular, the elastomer 14 may be embedded within the textile 16, for example, by sewing, gluing, or other fastening or attachment methods. The textile 16 may include, but is not limited to, a bed sheet or other flexible fabric, such as a garment, or may be a patch with an adhesive configured to adhere to the skin of the body the living being. For example, the textile 16 may be an ECG electrode patch used for measuring electrical signals of the heart and the elastomer 14 may be embedded therein and use a common power source. The textile 16 may further be apparel, such as a shirt, wrap, pants, underwear, a waist band, or may be a pillow case, blanket or any textile that is flexible, for example, polyamide fibers. The textile 16 may define a thickness of 0.05 to 7 mm as to maintain the sensitivity of the sensor 12 without direct contact with the living being when in use.

In the configuration shown in FIGS. 1 and 2, the elastomer 14 defines an undulating shape and is sandwiched between a first layer of material 18 and a second layer of material 20 of the textile 16 such that the textile surrounds or otherwise encloses the elastomer 14. In another configuration, the elastomer 14 defines a mat or mesh substantially commensurate in area as the area of the textile 16. In another configuration, the elastomer 14 defines a star shape or a serpentine shape to span a predefined of the area of the textile 16. In another configuration, the elastomer 14 may be wound like a coil around individual or a plurality of fibers that encompass the textile 16, or vice versa. In certain configurations, the elastomer 14 may define a shape that is commensurate or corresponds to a particular portion of the body of the living being to be monitored. For example, in one configuration, the elastomer 14 may be included within upholstery or fabric of a wheel chair or automobile seat. In such configurations, the elastomer 14 may be arranged to approximate the shape of living being's legs or buttocks.

Continuing to refer to FIG. 1, the elastomer 14 may define a first end 22 and a second end 24 opposite the first end 22. In one configuration, the first end 22 and the second end 24 are disposed proximate each other such that they be combine within a conductive electrical connector 26. That is, in one configuration, the electrical connector 26 may be configured to engage the first end 22 with the second end 24 to define a closed loop or a substantially closed with the electrical connector 26 such that resistance changes in the elastomer 14 may be measured. The electrical connector 26 may be configured to releasably engage with a controller 28 of system 10 configured to measure, in real time, changes in electrical resistance of the elastomer 14 owing to a deformation force applied to the elastomer 14. In the configuration shown in FIG. 1, the controller 28 is remote from the textile 16 and the elastomer 14. In one such configuration, a cable 30 may connect the electrical connector 26 to the controller 28 to maintain the controller 28 a distance away from the elastomer 14 and the textile 16. For example, when the sensor 12 is utilized to monitor movements of an infant, maintaining the controller 28 at a remote distance from the sensor 12 may prevent inadvertent disconnecting of the controller 28 from the sensor 12 and may further prevent unwanted wireless transmissions proximate to the infant. In another configuration, the electrical connector 26 may include a wireless transmitter/receiver to communicate with the controller 28 without a cable 30. In other configurations, the controller 28 may be coupled directly or indirectly to the textile 16, for example, by being affixed, embedded, or otherwise included on the textile 16 and coupled to the elastomer 14. In such a configuration, the controller 28 may be a MEMS device having an integrated battery configured to be charged inductively or through radiofrequency and may be directly connected to the elastomer 14.

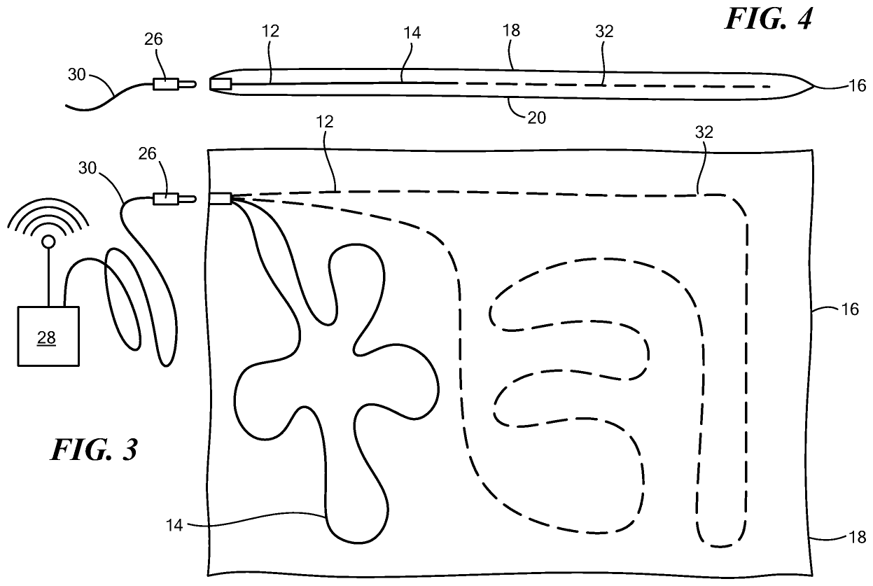

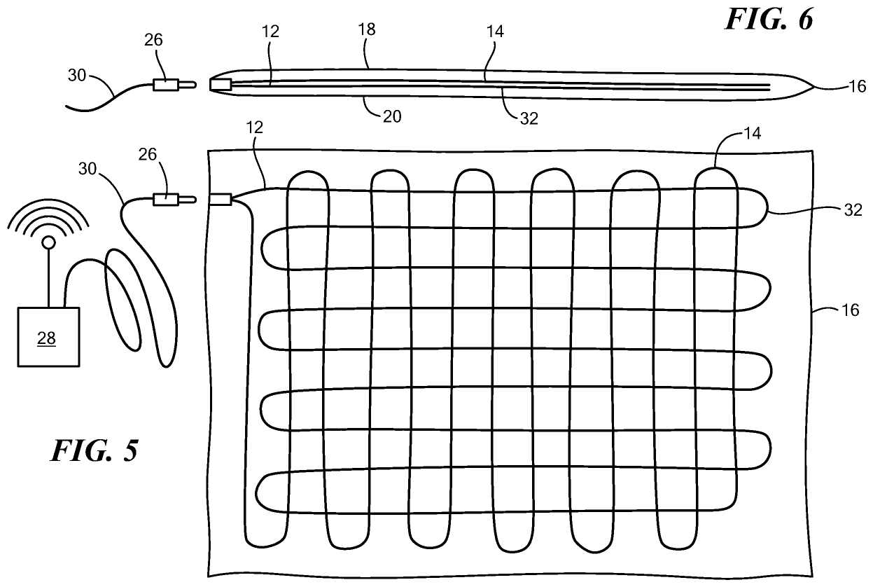

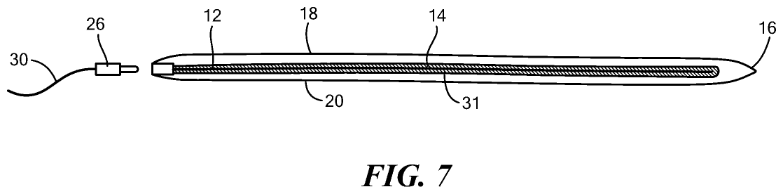

Referring now to FIGS. 3-6, the sensor 12 may include one or more conductive elastomers 14 engaged to the textile 16 as discussed above. For example, a second conductive elastomer 32 may be disposed within the textile 16 and in communication with the controller 28 through either the same electrical connector 26 or a different connector. Although two elastomers 14 and 32 are shown in FIG. 3, any number of conductive elastomers are contemplated by this disclosure to be engaged to the textile 16. In one configuration, as shown in FIGS. 3-4, each the elastomer 14 and the second conductive elastomer 32 are aligned along the same plane defined by the textile 16. In particular, the textile 16 may define a length, a width, and a height. As shown in FIG. 4, the elastomer 14 and the second conductive elastomer 32 may be disposed at the same height within the textile 16, but do not occupy the same area. In other configurations, as shown in FIGS. 5-6, the elastomer 14 and the second conductive elastomer 32 are disposed at different heights, but occupy the same length and width of the textile 16. In such configurations, each elastomer 14 or 32 may have the same variable resistances and sensitivities or different variable resistances and different sensitives. For example, elastomer 14 may be configured and calibrated to measure changes in resistance between a certain predetermined range of resistances that may correspond to particular movements, for example, breathing or movement, and elastomer 32 may be configured and calibrated to measure changes in resistance between a certain predetermined ranges of resistances that correspond to other movements. For example, body movements associated with breathing may be different than movement of appendages and may be detected by different elastomers within the sensor 12 and some elastomers 14 or 32 may be aligned to detect movements generated by the heart, to detect the heartbeat, or cardiac coherence. Accordingly, the sensor 12 may be designed and configured with an associated textile 16 depending on its particular use as discussed in more detail below. Optionally, as shown in FIG. 7, the elastomer 14 is surrounded, enclosed, or coated with a non-conductive element 31, for example, silicon to avoid any direct skin-to-sensor contact.

The controller 28 may include one or more processors and processing circuitry configured to carry out programmed instructions. For example, the controller 28 may be configured to measure changes in the resistance of the conductive elastomer 14 and/or conductive elastomer 32 in real time and correlate the measured changes in the resistance of the conductive elastomer 14 and/or conductive elastomer 32 to either breathing or movement of the living being when at least a portion of the body of the living being applies a force to the textile 16 without direct contact to the sensor 12. In one configuration, the controller 28 includes one or more Wheatstone bridges to measure changes in resistance, but other electrical circuits known in the art for measuring resistances may be included, for example, differentiation detectors and amplifiers.

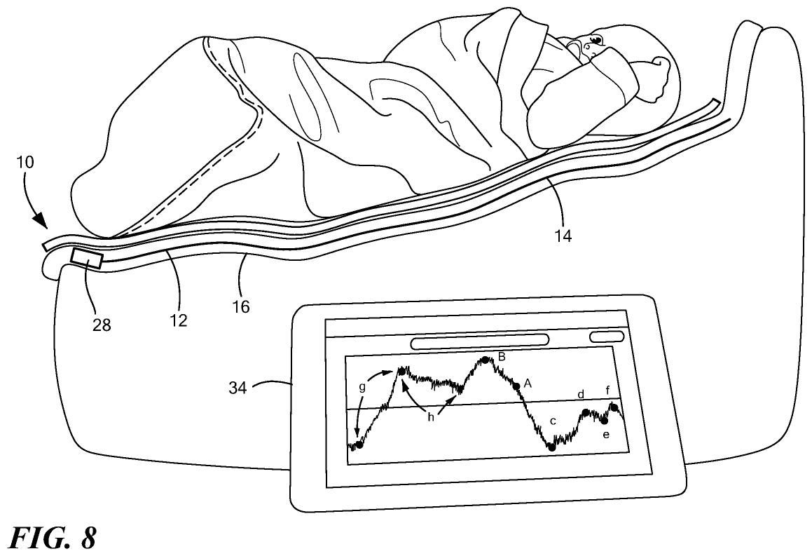

Referring now to FIGS. 8-10, in an exemplary use of system 10, an infant is shown positioned on top of the sensor 12 which is disposed within textile 16. The infant may be wearing clothes and have a blanket and/or pouch disposed around the infant's body, which in one example, may include up to 2 cm or more of textiles. Disposed underneath the blanket and/or pouch may be the textile 16 with the sensor 12 engaged thereto and/or therein. In this configuration, the controller 28 is integrated into the textile 16 and is coupled to the elastomer 14. The controller 28 may further communicate wirelessly, by Bluetooth, or other coupling methods known in the art, with a remote computing device 34, for example, an iPhone® or iPad®, Smartwatch, Smartphone, tablet, or other computing device having a display and a processing circuit configured to display data received from the controller 28. As shown in FIG. 8, the changes in resistance data measured by the controller 28 are displayed on the remote computing device 34 in real time. In particular, as the infant moves, a signal trace corresponding to the change in resistance data is displayed. Owing to the sensitivity of the sensor 12, voluntary and non-rhythmic body movements of the infant, for example, crying or shaking, may be distinguished from involuntary and rhythmic movements such as breathing. The signal trace showing data points from A to B represent the arms moving of the infant and data points c to d represent the infant breathing in and breathing out, respectively. That is, small amplitude changes are indicative of the infant's respiration and larger amplitudes are indicative of movement. In particular, as a non-limiting examples, breathing may occur with a frequency between 0.1 and 1.2 Hz with a high amplitude; arterial blood flood may occur at 0.5 to 4 Hz with a low amplitude; body movements between 0 to 2 Hz with a high amplitude; nervous movements can peak between 5 and 100 Hz; death would have no movement; movements associated with speaking between 10 to 30 Hz; and venous blood flood may occur from 0.01 Hz to 3 Hz with a high amplitude.

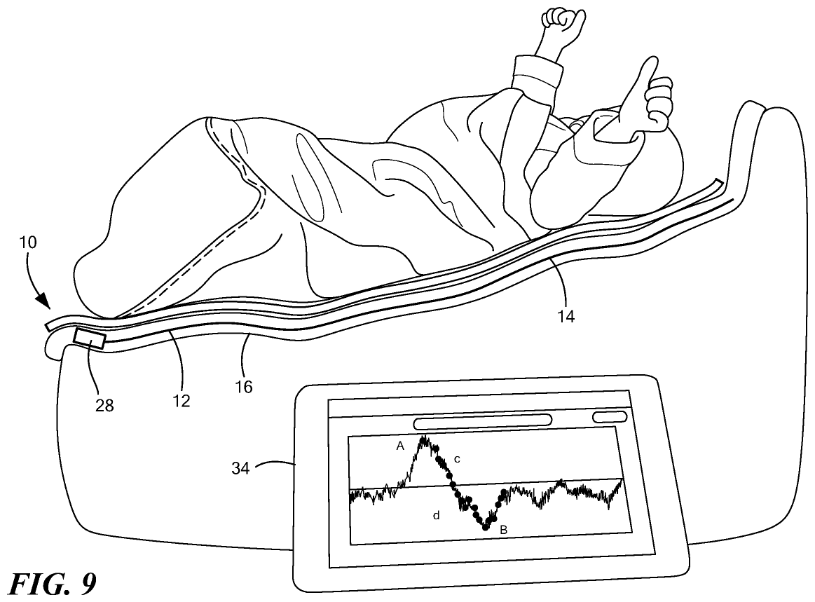

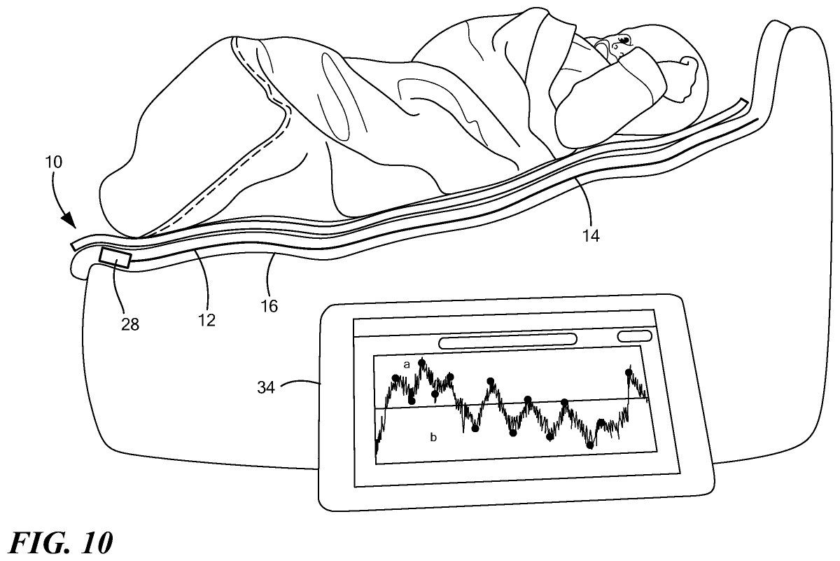

As shown in FIG. 9, the amplitude of the signal trace is indicative of the agitation level and rhythm of the infant's movement. For example, data points from g to h, h to B, and B to A are indicative of movement and agitation, whereas points c to d, d to e, and e to f are indicative of breathing. FIG. 10 shows an example of the infant at rest and exhibiting a normal breathing pattern of points a to b of respiration. The controller 28 or the computing device 34 may further include processing circuitry configured to execute an algorithm to determine one or more breathing and/or movement conditions based on the measured changes in resistance. For example, in the examples shown in FIGS. 8-10, the algorithm may assign a value to the amplitude of the infant's movements, a value to the pattern of the infant's movements, and a value the respiration pattern and correlate those values to predetermined values or parameters. For example, when the infant is not breathing or moving, all three of those values will be zero, for example, in the case of SIDS. Non-limiting examples of breathing patterns the controller 28 or the computing device 34 may be programmed to detect include normal awake breathing, normal asleep breathing, breathing coherence, thoracoabdomnal paradox, Kussmaul's breathing, apneustic breathing, Cheyne-Stokes respiration, atacix breathing, Biot's breathing, and central apnea.

The algorithm may further be programmed with a predetermined normal awake or non-awake breathing patterns that the controller 28 or computing device 34 may correlate to the measured resistance changes of the person or animal wearing or otherwise engaged to the textile 16 including the sensor 12. If the pattern of the measured resistance, or the value subscribed thereto, deviates by a predetermined threshold value from the predetermined normal awake or non-awake pattern, for example, by 5-30%, then an alert may be generated. Alternatively, if the measured changes in resistances, or the value subscribed thereto, matches a pre-programmed breathing or movement pattern or value corresponding to an undesirable pattern or value, the alert may also be generated. The alert may include, but is not limited to, a visual, audible, and/or tactile alert. For example, in the configuration in which the sensor 12 is included in upholstery of a seat in a commercial truck, the controller 28 and/or the sensor 12 may include haptic feedback to wake the driver when the algorithm determines that the driver's breathing or movement pattern corresponds to a condition of being asleep, the sensor 12 and/or the controller 28 may vibrate to awake the drive and/or generate an audible alter to awake the driver.

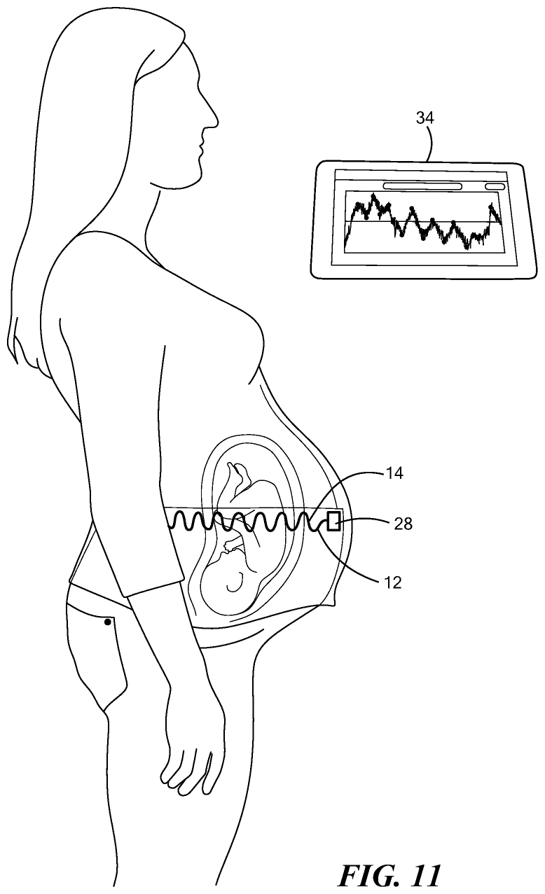

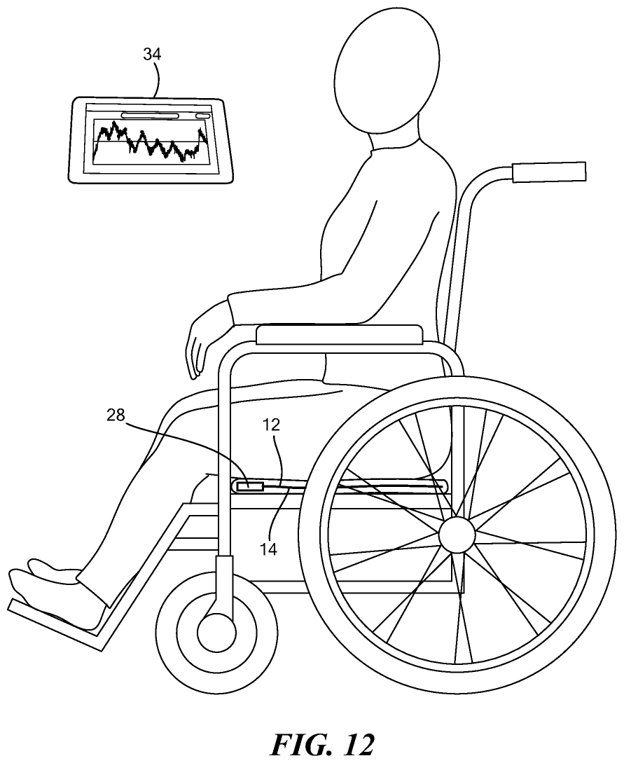

Referring back now to FIG. 11, the sensor 12 may be engaged to the textile 16 in any of the configurations described above, and the textile 16 may be placed over the abdomen of a living being, or under the living being's back, or any position on the living being's body for the desired measurement. In one configuration, the textile 16 is shirt with the sensor 12 integrated therein and surrounds the person's abdomen. In one example, the textile 16 may be a patch, shirt, pants, or an undergarment in which the sensor 12 is integrated with and configured to be disposed on the abdomen of a pregnant woman. For example, the elastomer 14 may be embedded within the waistband of undergarment or pants at least partially surrounding the person's waist such that it is proximate the abdomen of the pregnant woman without direct contact to the skin. In the configuration shown in FIG. 11, the sensor 12 is embedded within underwear of the person. The sensor 12 may be configured to measure changes in resistance associated with fetal movements, in addition to or excluding the movements of the person wearing the sensor 12, which may eliminate the need for large and bulky fetal monitors used in hospitals, and may further be used for ambulatory patients. For example, the sensor 12 may detect pregnancy contractions, in addition to fetal movements, and the controller 28 and/or the computing device 34 may display those movements in real time and distinguish between the two to anticipate labor and delivery times. In such a configuration, the controller 28 may be integrated with the textile 16. As in other configurations described above, measured fetal movements and contractions may be correlated to predetermined movements associated with the position of the fetus, growth of the fetus, and other in utero conditions, and alerts may be generated when the movement patterns deviate from normal parameters or when the movement patterns match known abnormal movements. In another example, as shown in FIG. 12, the sensor 12 may embedded within a wheelchair seat or in the form of a cushion or seat disposed on the wheel chair seat. In such a configuration the elastomer 14 may define a pattern that matches the buttocks or the upper thighs of the patient such that changes in resistance of the elastomer 14 may be correlated to movement to determine if the person is awake or asleep.

Other non-limiting examples of fields of use contemplated by this disclosure that may use system 10 include sleep apnea detection, by integrating the sensor 12 into a patch on the skin of the patient's chest or with a textile 16 to monitor breathing patterns; stress detection by integrating the sensor 12 into clothing; sleep onset detection by embedding the sensor within upholstery, wheelchair seats, sheets, or other textiles 16 discussed above; local plethysmography; and emotional status detection, for example, by embedding the sensor 12 within a shirt and measuring breathing patterns and voice intensity.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A device for monitoring breathing or movement of a living being, comprising:
a sensor, the sensor including a conductive elastomer having a variable resistance; and
a textile engaged to the sensor.

2. The device of claim 1, wherein the conductive elastomer planar.

3. The device of claim 1, wherein the conductive elastomer is embedded within the textile.

4. The device of claim 1, wherein the sensor defines a first end and a second end opposite the first end, and wherein the sensor includes an electrical connector, and wherein the first end and the second end are disposed within the electrical connector.

5. The device of claim 4, wherein the sensor includes a second conductive elastomer having a variable resistance different than the conductive elastomer.

6. The device of claim 5, wherein the textile defines a length, width, and height, and wherein the conductive elastomer and the second conductive elastomer are each at least one from the group consisting of:
disposed entirely at the same height within the textile; and
disposed at entirely at different heights within the textile.

7. The device of claim 1, further including a non-conductive material enclosing the conductive elastomer.

8. The device of claim 1, wherein the textile is at least one form the group consisting of a garment, a bed sheet, and a patch including an adhesive configure to be removeably adhered to skin of the living being.

9. The device of claim 1, wherein the variable resistance of the conductive elastomer is between 1 kohms and 100 kohms.

10. The device of claim 1, wherein the conductive elastomer changes resistance when the conductive elastomer is deformed.

11. A medical system for monitoring breathing or movement of a living being having a body, comprising:
a sensor, the sensor including a conductive elastomer having a variable resistance;
a textile engaged to the sensor;
a controller in communication with the sensor, the controller being configured to, in real time:
measure changes in a resistance of the conductive elastomer; and
correlate the measured changes in the resistance of the conductive elastomer to at least one from the group consisting of breathing and movement of the living being when at least a portion of the body of the living being applies a force to the textile without direct contact to the sensor.

12. The system of claim 11, wherein the controller is further configured to:
identify a breathing pattern based on the measured changes in the resistance of the conductive elastomer; and
compare the identified breathing pattern to a plurality predetermined abnormal breathing patterns; and
if the identified breathing pattern corresponds to one of the plurality of predetermined abnormal breathing patterns, generate an alert.

13. The system of claim 11, wherein the controller includes a wireless communication transmitter/receiver configured to communicate with a remote controller.

14. The system of claim 11, wherein the conductive elastomer is planar.

15. The system of claim 11, wherein the variable resistance of the conductive elastomer is between 1 kohms and 100 kohms.

16. The system of claim 11, wherein the sensor define a first end and a second end opposite the first end, and wherein the sensor includes an electrical connector, and wherein the first end and the second end are disposed within the electrical connector.

17. The system of claim 16, wherein the sensor includes a second conductive elastomer having a variable resistance different than the conductive elastomer.

18. The system of claim 11, wherein the controller is integral with the textile.

19. The system of claim 11, wherein the textile is at least one form the group consisting of a garment, a bed sheet, and a patch including an adhesive configure to be removeably adhered to skin of the living being.

20. A medical system for monitoring breathing or movement of a living being having a body, comprising:
a sensor including a conductive elastomer having a variable resistance between 1 kohms and 100 kohms;
a textile, the sensor being enclosed within the textile; and
the sensor defining a first end and a second end opposite the first end, the sensor includes an electrical connector extending away from the textile, and the first end and the second end are disposed within the electrical connector.
a controller in communication with the sensor and configured to receive the electrical connector, the controller being configured to, in real time:
measure changes in a resistance of the conductive elastomer;
correlate the measured changes in the resistance of the conductive elastomer to at least one from the group consisting of breathing and movement of the living being when the body of the living being applies a force to the textile without direct contact to the sensor;
identify a breathing pattern based on the measured changes in the resistance of the conductive elastomer;
compare the identified breathing pattern to a plurality predetermined abnormal breathing patterns; and
if the identified breathing pattern corresponds to one of the plurality of predetermined abnormal breathing patterns, generate an alert.
